(19) United States
(12) Patent Application Publication
(10) Pub. No.: US 2023/0402182 A1
(43) Pub. Date: Dec. 14, 2023

(54) MACHINE LEARNING BASED CLINICAL RESOURCE CONTROLLER

(71) Applicant: CAREFUSION 303, INC., San Diego, CA (US)

(72) Inventors: Abhikesh Nag, San Diego, CA (US); Lyazzat Segizbayeva, San Diego, CA (US); Latha Vankeepuram, San Diego, CA (US); Ying Tabak, Marlborough, MA (US)

(21) Appl. No.: 18/236,374

(22) Filed: Aug. 21, 2023

Related U.S. Application Data
(63) Continuation of application No. PCT/US2022/013932, filed on Jan. 26, 2022.
(60) Provisional application No. 63/152,318, filed on Feb. 22, 2021.

(51) Int. Cl. G16H 50/20

(52) U.S. Cl. CPC G16H 50/20

(57) ABSTRACT

A method may include receiving, from one or more data systems, a message. A machine learning model may be applied to the message to determine whether the message is an actionable message or a non-actionable message. In response to the message being the actionable message, the machine learning model may be applied to extract, from the message, a clinically significant data. One or more tasks may be performed based on the clinically significant data. The one or more tasks may include performing, based on the clinically significant data, a resource allocation for a clinical workflow associated with the data systems. The one or more tasks may also include detecting systematic inefficiencies and bottlenecks associated with the clinical workflow. Related methods and articles of manufacture, including computer program products, are also disclosed.

### CROSS-REFERENCE TO RELATED APPLICATIONS

This application is a continuation of International Application No. PCT/US2022/013932, entitled “MACHINE LEARNING BASED CLINICAL RESOURCE CONTROLLER” and filed Jan. 26, 2022, which claims the benefit of and priority to U.S. Provisional Application No. 63/152,318, entitled “MACHINE LEARNING BASED CLINICAL RESOURCE CONTROLLER” and filed on Feb. 22, 2021, the disclosure of each of which is incorporated herein by reference in its entirety.

### TECHNICAL FIELD

The subject matter described herein relates generally to machine learning and more specifically to a machine learning based technique for resource management in a clinical and diagnostic setting.

### BACKGROUND

Laboratory automation is a multi-disciplinary effort to research and develop technologies to optimize the speed, efficiency, and outcome of laboratory workflows. For example, a laboratory information system (LIS) may include hardware and software configured to provide support for laboratory activities such as inoculation, incubation, plate imaging, culture reading, result reporting, and/or the like. The laboratory information system may record, analyze, store, and share data generated by various laboratory activities. In doing so, the laboratory information system may aim to deliver timely, accurate, and relevant information, whether in clinical settings where the focus tends to be on patient-specific specimen or in non-clinical settings such as research laboratories and/or the like.

### SUMMARY

Systems, methods, and articles of manufacture, including computer program products, are provided for a machine learning based resource controller. In some example embodiments, there is provided a system that includes a machine learning model trained to receive, as at least one input value, one or more messages and generate at least one output value indicating whether the one or more messages are actionable. The system may further include at least one processor and at least one memory. The at least one memory may include program code that provides operations when executed by the at least one processor. The operations may include: receiving, from one or more data systems, a message for a patient; determining, using the machine learning model and the message, that the message is actionable; extracting, from the message, a clinically significant data; and controlling, based at least on the clinically significant data, at least one medical device associated with the patient to perform one or more tasks.

In some variations, one or more features disclosed herein including the following features can optionally be included in any feasible combination. The performing of the one or more tasks may include identifying, based at least on the clinically significant data, a stage of a clinical workflow associated with the one or more messages, determining, based at least on a timestamp associated with the one or more messages, a quantity of time between two or more successive stages of the clinical workflow, and in response to the quantity of time between the two or more successive stages of the clinical workflow exceeding a threshold value, determining one or more corrective configurations for the at least one medical device.

In some variations, the one or more corrective actions may include modifying a scheduling of one or more activities associated with the clinical workflow and/or adjusting an allocation of resources associated with the one or more activities.

In some variations, the clinical workflow may include a microbial testing workflow and/or a virology assay.

In some variations, the stage of the clinical workflow may include a start of a culturing process for a microbe, a gram positive or gram negative identification for the microbe, a species and/or organism identification for the microbe, or an antimicrobial susceptibility of the microbe.

In some variations, the performing of the one or more tasks may include determining, based at least on the clinically significant data, an allocation of resources at the one or more data systems.

In some variations, the allocation of resources may include allocating a resource in response to a result of a clinical workflow being associated with the resource.

In some variations, the resource may include an antimicrobial based at least on the result of the clinical workflow indicating a presence of a microbe susceptible to the antimicrobial.

In some variations, the allocation of the resources may include determining, based at least on the clinically significant data, a subsequent stage of a clinical workflow and a time for the subsequent stage of the clinical workflow, and scheduling, in accordance with the time of the subsequent stage of the clinical workflow, a quantity of resources required for the subsequent stage of the clinical workflow.

In some variations, the machine learning model may include a regression model, an instance-based model, a regularization model, a decision tree, a Bayesian model, a clustering model, an associative model, a neural network, a deep learning model, a dimensionality reduction model, and/or an ensemble model.

In some variations, the clinically significant data may be extracted by at least applying, to the message, the machine learning model and/or a different machine learning model.

In some variations, the machine learning model may be trained to identify and tag the clinically significant data included in the message. The message may be determined to be actionable in response to more than a threshold quantity of data in the message being tagged as clinically significant.

In some variations, the machine learning model may be further trained to at least receive, as the at least one input value, a sequence of messages including the message, and generate the at least one output value to indicate whether the sequence of messages are actionable.

In some variations, the at least one output value may indicate that the message is associated with a first actionable event. The at least one output value may further indicate that sequence of messages are associated with a second actionable event.

In some variations, the machine learning model may determine the message to be actionable as part of the sequence of messages.

In some variations, the machine learning model may include a recurrent neural network (RNN), a hidden Markov model, a conditional random field (CRF) model, and/or a gated recurrent unit (GRU).

In some variations, the at least one medical device may include a diagnostic device, an infusion pump, a dispensing cabinet, and/or a wasting station.

In some variations, the controlling of the at least one medical device may include transmitting, to the at least one medical device, one or more messages to adjust an operational state and/or a functional element of the at least one medical device.

In some variations, the one or more messages may include one or more instructions, which when executed by a processor associated with the at least one medical device, adjust the operational state and/or the functional element of the at least one medical device.

In some variations, the one or more messages may include one or more values, which when applied at the at least one medication device, adjust the operational state and/or the functional element of the at least one medical device.

In another aspect, there is provided a method for machine learning based safety controls. The method may include: receiving, from one or more data systems, a message for a patient; determining, using a machine learning model and the message, that the message is actionable, the machine learning model being trained to receive, as at least one input value, the message and generate at least one output value indicating whether the message is actionable; extracting, from the message, a clinically significant data; and controlling, based at least on the clinically significant data, at least one medical device associated with the patient to perform one or more tasks.

In some variations, one or more features disclosed herein including the following features can optionally be included in any feasible combination. The performing of the one or more tasks may include identifying, based at least on the clinically significant data, a stage of a clinical workflow associated with the one or more messages, determining, based at least on a timestamp associated with the one or more messages, a quantity of time between two or more successive stages of the clinical workflow, and in response to the quantity of time between the two or more successive stages of the clinical workflow exceeding a threshold value, determining one or more corrective configurations for the at least one medical device.

In some variations, the one or more corrective actions may include modifying a scheduling of one or more activities associated with the clinical workflow and/or adjusting an allocation of resources associated with the one or more activities.

In some variations, the clinical workflow may include a microbial testing workflow and/or a virology assay.

In some variations, the stage of the clinical workflow may include a start of a culturing process for a microbe, a gram positive or gram negative identification for the microbe, a species and/or organism identification for the microbe, or an antimicrobial susceptibility of the microbe.

In some variations, the performing of the one or more tasks may include determining, based at least on the clinically significant data, an allocation of resources at the one or more data systems.

In some variations, the allocation of resources may include allocating a resource in response to a result of a clinical workflow being associated with the resource.

In some variations, the resource may include an antimicrobial based at least on the result of the clinical workflow indicating a presence of a microbe susceptible to the antimicrobial.

In some variations, the allocation of the resources may include determining, based at least on the clinically significant data, a subsequent stage of a clinical workflow and a time for the subsequent stage of the clinical workflow, and scheduling, in accordance with the time of the subsequent stage of the clinical workflow, a quantity of resources required for the subsequent stage of the clinical workflow.

In some variations, the machine learning model may include a regression model, an instance-based model, a regularization model, a decision tree, a Bayesian model, a clustering model, an associative model, a neural network, a deep learning model, a dimensionality reduction model, and/or an ensemble model.

In some variations, the clinically significant data may be extracted by at least applying, to the message, the machine learning model and/or a different machine learning model.

In some variations, the machine learning model may be trained to identify and tag the clinically significant data included in the message. The message may be determined to be actionable in response to more than a threshold quantity of data in the message being tagged as clinically significant.

In some variations, the machine learning model may be further trained to at least receive, as the at least one input value, a sequence of messages including the message, and generate the at least one output value to indicate whether the sequence of messages are actionable.

In some variations, the at least one output value may indicate that the message is associated with a first actionable event. The at least one output value may further indicate that sequence of messages are associated with a second actionable event.

In some variations, the machine learning model may determine the message to be actionable as part of the sequence of messages.

In some variations, the machine learning model may include a recurrent neural network (RNN), a hidden Markov model, a conditional random field (CRF) model, and/or a gated recurrent unit (GRU).

In some variations, the at least one medical device may include a diagnostic device, an infusion pump, a dispensing cabinet, and/or a wasting station.

In some variations, the controlling of the at least one medical device may include transmitting, to the at least one medical device, one or more messages to adjust an operational state and/or a functional element of the at least one medical device.

In some variations, the one or more messages may include one or more instructions, which when executed by a processor associated with the at least one medical device, adjust the operational state and/or the functional element of the at least one medical device.

In some variations, the one or more messages may include one or more values, which when applied at the at least one medication device, adjust the operational state and/or the functional element of the at least one medical device.

In another aspect, there is provided a computer program product that includes a non-transitory computer readable medium storing instructions. The instructions may cause operations when executed by at least one data processor. The operations may include: receiving, from one or more data systems, a message for a patient; determining, using a machine learning model and the message, that the message is actionable, the machine learning model being trained to receive, as at least one input value, the message and generate at least one output value indicating whether the message is actionable; extracting, from the message, a clinically significant data; and controlling, based at least on the clinically significant data, at least one medical device associated with the patient to perform one or more tasks.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to applying a machine learning model to manage resources in a clinical and diagnostic setting, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a clinical management system, in accordance with some example embodiments;

FIG. 2 depicts a flowchart illustrating an example of a process for machine learning based message parsing, in accordance with some example embodiments;

FIG. 3A depicts a flowchart illustrating an example of a process for machine learning based clinical workflow analysis, in accordance with some example embodiments;

FIG. 3B depicts a flowchart illustrating an example of a process for machine learning based resource allocation, in accordance with some example embodiments;

FIG. 4 depicts a block diagram illustrating a computing system, in accordance with some example embodiments; and

FIG. 5 depicts an example of unstructured data, in accordance with some example embodiments.

When practical, similar reference numbers denote similar structures, features, or elements.

### DETAILED DESCRIPTION

A laboratory information system (LIS) may be deployed in clinical and non-clinical settings to support various laboratory activities such as inoculation, incubation, plate imaging, culture reading, result reporting, and/or the like. The laboratory information system may record, analyze, store, and share data arising from such laboratories activities. For example, the laboratory information system may generate a variety of messages at each stage of a microbial test, virology assay, and/or the like. Some messages may convey clinically significant data and are therefore actionable messages associated with additional tasks while others, such as transitional status messages and/or the like, are non-actionable messages. Moreover, these messages may include unstructured data that varies across different medical devices, clinical facilities, and automation platforms. Inconsistency in the content and format of the messages may thwart efforts to establish a uniform data interface across devices, facilities, and platforms. This lack of interoperability may compromise the speed, efficiency, and outcome of various clinical workflows. Downstream decisions, such as those involving workflow statistics and strategic resource allocation, may suffer in the absence of timely, accurate, and relevant information from the laboratory information system.

In some example embodiments, a message exchange may receive messages from a variety of sources across different medical devices, clinical facilities, and automation platforms. To provide a uniform data interface, the message exchange may include one or more machine learning models trained classify messages containing unstructured data. For example, the message exchange may receive messages from a laboratory information system (LIS) engaged in a workflow such as microbial testing, virology assay, and/or the like. The one or more machine learning models may be trained to identify actionable messages and extract clinically significant data to enable downstream tasks and decisions such as the collection of workflow statistics, resource allocation, and/or the like.

For the microbial testing workflow, the machine learning model may identify actionable messages indicating a start of a culturing process for a microbe, a gram positive or gram negative identification for the microbe, a species and/or organism identification for the microbe, an antimicrobial susceptibility of the microbe, and/or the like. Moreover, the machine learning model may extract, from each actionable message, clinically significant data to enable downstream clinical actions and decisions. For example, the output of the machine learning model may include the identification of cultural isolates and the results of antimicrobial susceptibility tests (AST), which may form the basis of downstream decisions in resource allocation, treatment, and/or the like. The availability of timely, accurate, and relevant information from the laboratory information system may expedite the clinical actions needed to render effective antimicrobial treatment, reduce antimicrobial resistance risk, and improve patient outcome.

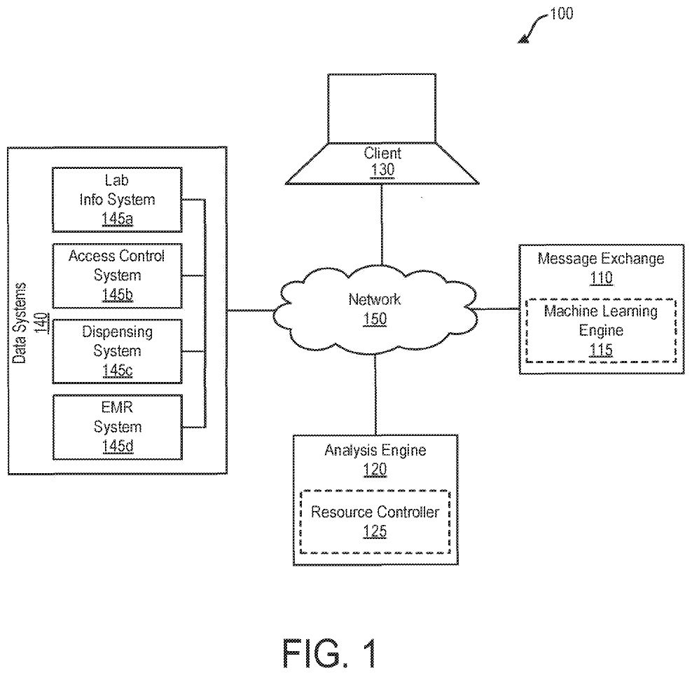

FIG. 1 depicts a system diagram illustrating an example of a clinical management system 100, in accordance with some example embodiments. Referring to FIG. 1, the clinical management system 100 may include a message exchange 110, an analysis engine 120 including a resource controller 125, a client 130, and one or more data systems 140. As FIG. 1 shows, the message exchange 110, the analysis engine 120, the client 130, and the one or more data systems 140 may be communicatively coupled via a network 150. The client 130 may be a processor-based device including, for example, a smartphone, a tablet computer, a wearable apparatus, a desktop computer, a laptop computer, a workstation, and/or the like. Meanwhile, the network 150 may be any wired and/or wireless network including, for example, a public land mobile network (PLMN), a local area network (LAN), a virtual local area network (VLAN), a wide area network (WAN), the Internet, and/or the like.

The message exchange 110 and the analysis engine 120 may be accessible to the client 130 as a cloud-based service (e.g., a software-as-a-service (SaaS) and/or the like). Alternatively, the message exchange 110 and/or the analysis engine 120 may be at least partially embedded and/or implemented within the one or more data systems such as, for example, at a laboratory information system (LIS) 145a, an access control system 145b, a dispensing system 145c, an electronic medical record (EMR) system 145d, and/or the like. That is, the message exchange 110 and/or the analysis engine 120 may be at least partially embedded and/or implemented within a medical device such as, for example, a dispensing cabinet, an infusion pump, a wasting station, and/or the like. For example, in some example embodiments, the message exchange 110 may be a centralized, cloud-based service whereas the analysis engine 120 may be deployed across the data systems 140. Accordingly, at least some functionalities of the message exchange 110 and/or the analysis engine 120 may be accessed locally at the one or more data systems 140. Moreover, the message exchange 110 and/or the analysis engine 120 may be updated and/or configured as part of servicing and/or updating the corresponding data systems 140.

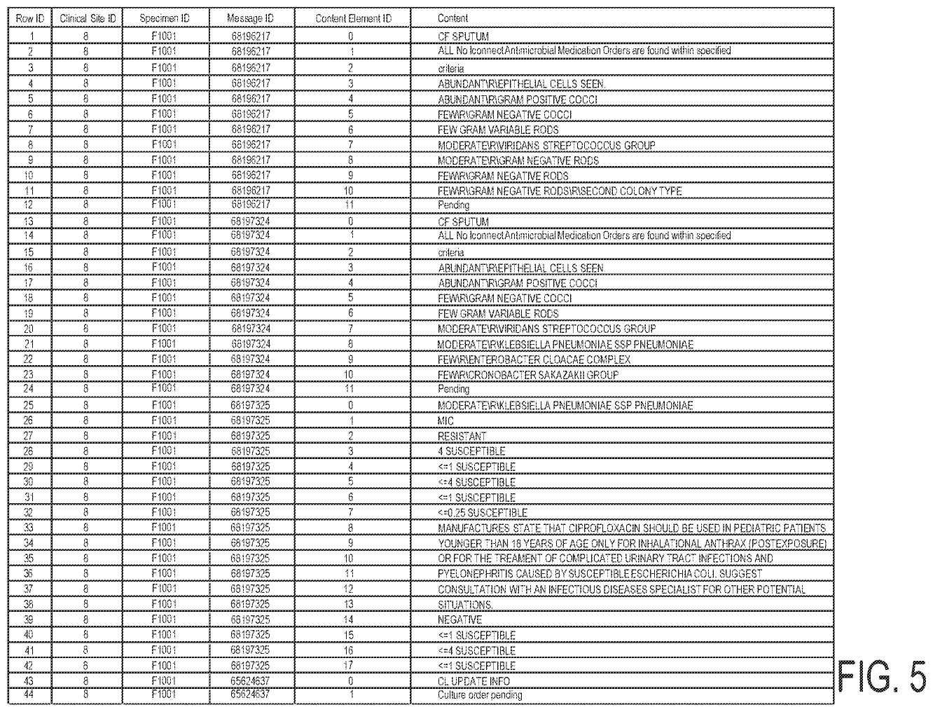

The message exchange 110 may receive messages generated by the one or more data systems 140 including, for example, the laboratory information system 145a, the access control system 145b, the dispensing system 145c, the electronic medical record system 145d, and/or the like. Some messages may convey clinically significant data and are therefore actionable messages associated with additional tasks while others, such as transitional status messages and/or the like, are non-actionable messages. Moreover, these messages may include unstructured data whose format and/or content may vary across different medical devices, clinical facilities, and automation platforms. To further illustrate, an example of unstructured data forming the messages generated by the one or more data systems 140 is shown in FIG. 5.

FIG. 5 shows table including unstructured data that may be processed using the systems, devices, or methods described. The table 500 may include multiple columns. As shown in FIG. 5, the columns are “Row ID”, “Clinical Site ID”, “Specimen ID”, “Message ID”, “Content Element ID”, and “Content”. Fewer, additional, or alternative columns may be included. For example, a source system identifier column may be included to store an indicator associated with the information system that generated the message. As another example, time or date information may be stored in a timestamp column.

An initial set of messages may be annotated such by labeling messages or portions of messages. The labels may be applied manually to a training set of messages. In some implementations, the labels may be applied automatically such as through keyword matching, regular expressions, or using a machine learning model that accepts message content as input and generates one or more labels as outputs. Examples of labels include “Gram Positive/Negative Result”, “Antibiotic Susceptibility”, “Don't Know”, “Organism Detected”, “Initial Progress Update”, or “Other”. In some implementations, a message may be associated with more than one label. The available labels may be specified as a configuration of the system or trained into the system via the machine learning model.

Once a message is associated with a label, the features described can based clinical significance on the presence of one or more labels. Additional model training may be applied to assess clinical significance based on sequences of labels, timing of events associated with specific labels, and the like. In this manner, the system can efficiently filter messages as received from the source systems to expedite any needed clinical action. In some implementations, when an event having clinical significance is identified, the system may transmit an alert to one or more devices. In some implementations, the clinical significance may be acute to a specific clinical need. In such implementations, the system may transmit a control message to one or more clinical devices to cause administration of a clinical response or other workflow (e.g., administration of a drug from an infusion pump, dispensing of a drug from an automated dispensing equipment, performing an assay or other test via a networked diagnostic or analytical device) or cause the clinical device to prepare for a clinical response (e.g., adjust power state, connect to a network, configure operational parameter (e.g., login, assay selection, control variable (e.g., pump rate, dispense location, etc.))). In the example shown in FIG. 5, the message content associated with “Row ID” 43 and 44 may be informational content having no clinical significance as compared to the message content associate with “Row ID” 1 through 12 which include results from a Gram test.

As used herein, the terms “control” or “controlling” encompass a wide variety of actions. For example, “controlling” a device may include transmitting one or more messages to adjust an operational state or functional element of the device. The message may include specific instructions to be executed by a processor of the device to manifest the change. The “controlling” may include storing a value in a location of a storage device for subsequent retrieval by the device to be controlled, transmitting a value directly to the device to be controlled via at least one wired or wireless communication medium, transmitting or storing a reference to a value, and the like. For example, a control message may include a value to adjust a level of power from a power source of the controlled device. As another example, a control message may activate or deactivate a structural element of the controlled device such as a light, audio playback, a motor, a lock, a pump, a display, or other component of a device described herein. “Controlling” may include indirect control of the device by adjusting a configuration value used by the controlled device. For example, the control message may include a threshold value for a device characteristic (e.g., temperature, rate, frequency, etc.). The threshold value may be stored in a memory location and referred to by the controlled device during operation.

The message exchange 110 may, for example, receive messages from the laboratory information system 145a while the laboratory information system 145a is engaged in a workflow such as microbial testing, virology assay, and/or the like. For the microbial testing workflow, the laboratory information system 145a may generate one or more actionable messages indicating, for example, a start of a culturing process for a microbe, a gram positive or gram negative identification for the microbe, a species and/or organism identification for the microbe, an antimicrobial susceptibility of the microbe, and/or the like. In addition to the one or more actionable messages, the laboratory information system 145a may also generate non-actionable messages, transitional status messages, and/or the like. The content and/or format of the messages from the laboratory information system 145a may be different from the content and/or format of the messages generated by the other data systems 140, such as the access control system 145b, the dispensing system 145c, the electronic medical record (EMR) system 145d, and/or the like. Absent a uniform data interface between the data systems 140, critical data generated by one data system, such as the laboratory information system 145, may not be available to the other data systems in a timely manner. A lack of interoperability between the data systems 140 may impair the speed, efficiency, and outcome of various clinical workflows that may depend on high throughput interactions or systems that generate high volumes of data (e.g., hundreds or thousands of messages) that may not be processed in an actionable manner without the technical features described.

In some example embodiments, to provide a uniform data interface between the data systems 140, the message exchange 110 may include a machine learning engine 115 including one or more machine learning models trained to identify actionable messages and extract clinically significant data to enable downstream tasks and decisions. The machine learning engine 115 may operate on messages from the one or more data systems 140 regardless of variations in the content and format of messages originating from different medical devices, clinical facilities, and automation platforms. For the microbial testing workflow, for example, the machine learning engine 115 identify the one or more actionable messages generated by the laboratory information system 145a and extract clinically significant data, such as the identification of cultural isolates and the results of antimicrobial susceptibility tests (AST). The clinically significant data extracted from these actionable messages may be sent, for example, to the analysis engine 120, to support various downstream tasks and decisions such as the collection of workflow statistics, resource allocation, and/or the like.

Some messages may be identified as individually actionable. Alternatively and/or additionally, some messages may be considered actionable as part of a group of messages or a sequence of messages. For example, a single message indicating that a specimen encountered at the laboratory information system 145a lacks antibiotic susceptibility may not be actionable on its own (or may constitute one type of an actionable event). Contrastingly, a sequence of messages (e.g., a sequence of more than a threshold quantity of messages) indicating a lack antibiotic susceptibility associated with multiple specimens encountered at the laboratory information system 145a may constitute an actionable event (or a different type of actionable event). Accordingly, the machine learning engine 115 may be configured to operate on multiple messages in order to detect actionable events that occur across a group of messages, a sequence of messages, and/or the like. For instance, the machine learning engine 115 may include a machine learning model trained to operate on sequential data. Examples of such a machine learning model may include a recurrent neural network (RNN), a hidden Markov model, a conditional random field (CRF) model, a gated recurrent unit (GRU), and/or the like.

As noted, clinically significant data may be extracted from actionable messages in order to enable one or more downstream tasks. For example, the analysis engine 120 may identify, based at least on a timestamp associated with the actionable messages, bottlenecks present in the microbial testing workflow in which one or more laboratory activities (e.g., inoculation, incubation, plate imaging, culture reading, result reporting, and/or the like) are associated with an above threshold delay. The analysis engine 120 may determine one or more corrective actions to minimize the bottlenecks including, for example, modifying the scheduling of the laboratories activities, adjusting the allocation of resources associated with the laboratories activities, and/or the like. Alternatively and/or additionally, when the output of the machine learning engine 115 indicates the presence of a microbe susceptible to an antimicrobial, for example, in more than a threshold quantity of specimen encountered at the laboratory information system 145a, the resource controller 125 at the analysis engine 120 may determine to allocate a corresponding quantity of the antimicrobial (and/or other resources).

The machine learning engine 115 may, as noted, include one or more machine learning models trained to identify actionable messages and extract clinically significant data. Examples of machine learning models include a regression model, an instance-based model, a regularization model, a decision tree, a Bayesian model, a clustering model, an associative model, a neural network, a deep learning model, a dimensionality reduction model, an ensemble model, and/or the like. Moreover, the machine learning engine 115 may include a single machine learning model trained to identify actionable messages as well as extract clinically significant data. For example, the machine learning model may be trained to identify and tag clinically significant data, in which case an actionable message may be a message in which the machine learning model tags more than a threshold quantity of data as clinically significant whereas a non-actionable message may be a message in which the machine learning model does not tag more than a threshold quantity of data as clinically significant. Alternatively, the machine learning engine 115 may include multiple machine learning models such as a first machine learning model trained to identify actionable messages and a second machine learning model trained to extract clinically significant data. Accordingly, messages identified as being actionable by the first machine learning model may be passed to the second machine learning model for the extraction of clinically significant data.

The one or more machine learning models may be trained using training data that includes annotated messages including, for example, messages that have been labeled as actionable or non-actionable, messages that have been labeled with a corresponding stage of the clinical workflow, messages whose content has been tagged to indicate clinically significant data present in the messages, and/or the like. The annotated messages may provide ground truth labels and tags for a supervised learning process in which the one or more machine learning model are trained to identify actionable messages and extract clinically significant data.

For example, training a machine learning models may include minimizing an error in an output of the machine learning models, which may correspond to a difference between the labels the machine learning model assigns to an annotated message and the ground-truth label associated with the annotated message. The training may include determining a gradient of an error function (e.g., mean squared error (MSE), cross entropy, and/or the like) associated with the machine learning model and adjusting one or more weights applied by the machine learning model until the gradient of the error function converges to a threshold value (e.g., a local minimum and/or the like).

In some example embodiments, the one or more machine learning models may be trained to learn an ontology associated with the messages output by the data systems 140 including, for example, the laboratory information system 145a, the access control system 145b, the dispensing system 145c, the electronic medical record (EMR) system 145d, and/or the like. The ontology associated with the messages may define, for example, different categories of messages, the relationship between the different categories of messages, and the data that may be present in each category. By learning the ontology, the one or more machine learning model may be trained to identify messages from different stages of a clinical workflow and to extract the clinically significant data that may arise during each stage of the clinical workflow.

In the case of the microbial testing workflow, for example, a machine learning model may be trained to identify a message from the laboratory information system 145a as being associated with the start of the culturing process for a microbe, a gram positive or gram negative identification for the microbe, a species and/or organism identification for the microbe, an antimicrobial susceptibility of the microbe, and/or the like. Furthermore, the same machine learning model (or a different machine learning model) may be trained extract, from the messages generated by the laboratory information system 145a, clinically significant data such as the identification of cultural isolates and the results of antimicrobial susceptibility tests (AST). Because the ontology defines the relationship between different messages, by learning the ontology, the one or more machine learning models may also be trained to determine the sequence and timing of the messages originating from the laboratory information system 145a.

In some example embodiments, the analysis engine 120 may determine, based at least on the output of the machine learning engine 115, various workflow statistics. For the microbial testing workflow, for example, the workflow statistics may be determined based at least on a volume and/or a timing of the messages from various stages of the microbial testing workflow (e.g., start of the culturing process for a microbe, a gram positive or gram negative identification for the microbe, a species and/or organism identification for the microbe, an antimicrobial susceptibility of the microbe, and/or the like). Examples of clinical workflow statistics may include various metrics such as a turnaround time (TAT) indicating a distribution of time elapsed between successive stages of a workflow such as the microbial testing workflow associated with the laboratory information system 145a. Alternatively and/or additionally, the analysis engine 120 may identify trends and/or establish benchmarks to enable a comparison between different medical devices, clinical facilities, and automation platforms. These metrics, trends, and/or benchmarks may enable a detection of systematic inefficiencies and bottlenecks across devices, facilities, and automation platforms, which is otherwise unfeasible in the absence of the uniform data interface provided by the message exchange 110.

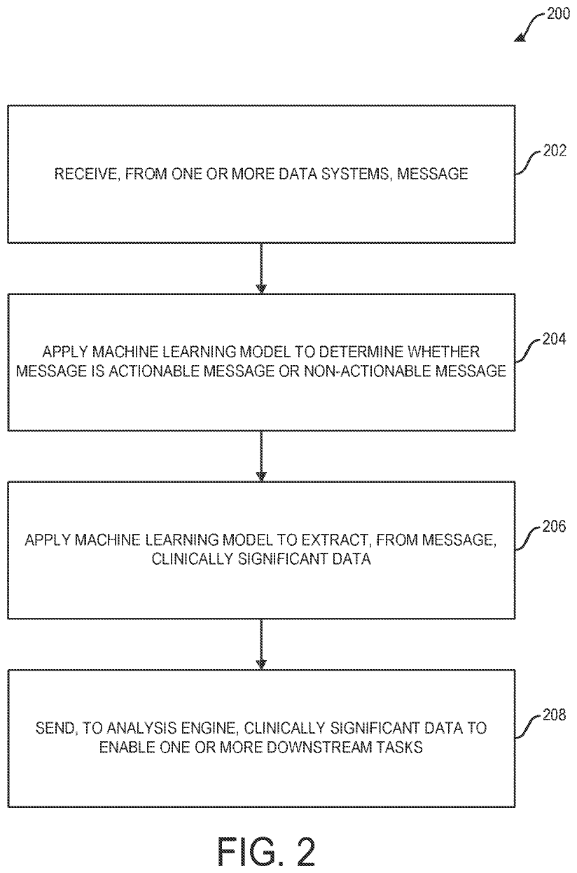

FIG. 2 depicts a flowchart illustrating an example of a process 200 for machine learning based message parsing, in accordance with some example embodiments. Referring to FIGS. 1-2, the process 200 may be performed by the message exchange 110 to parse messages generated, for example, by the one or more data systems 140 including the laboratory information system 145a, the access control system 145b, the dispensing system 145c, the electronic medical record system 145d, and/or the like.

At 202, the message exchange 110 may receive, from one or more data systems, a message. In some example embodiments, the message exchange 110 may receive messages generated by the one or more data systems 140 including the laboratory information system 145a, the access control system 145b, the dispensing system 145c, the electronic medical record system 145d, and/or the like. Some messages may convey clinically significant data and are therefore actionable messages associated with additional tasks while others, such as transitional status messages and/or the like, are non-actionable messages. Moreover, these messages may include unstructured data whose format and/or content may vary across different medical devices, clinical facilities, and automation platforms.

At 204, the message interface 110 may apply a machine learning model to determine whether the message is an actionable message or a non-actionable message. In some example embodiments, the machine learning engine 115 may include one or more machine learning models trained to differentiate between actionable messages conveying clinically significant data associated with additional tasks and non-actionable messages such as transitional status messages and/or the like. For example, the one or more machine learning models may be trained using annotated messages, which may include messages that have been labeled as actionable or non-actionable, messages that have been labeled with a corresponding stage of the clinical workflow, messages whose content has been tagged to indicate clinically significant data present in the messages, and/or the like. As such, the one or more machine learning models may be trained to learn an ontology associated with the messages output by the data systems 140 defining, for example, different categories of messages, the relationship between the different categories of messages, and the data that may be present in each category. By learning the ontology, the one or more machine learning models may be trained to identify messages from different stages of a clinical workflow and to extract the clinically significant data that may arise during each stage of the clinical workflow.

At 206, the message exchange 110 may apply a machine learning model to extract, from the message, clinically significant data. For example, for a microbial testing workflow, one machine learning model may be applied to identify a message from the laboratory information system 145a as being associated with the start of the culturing process for a microbe, a gram positive or gram negative identification for the microbe, a species and/or organism identification for the microbe, an antimicrobial susceptibility of the microbe, and/or the like. Furthermore, the same machine learning model (or a different machine learning model) may be applied to extract, from the messages generated by the laboratory information system 145a, clinically significant data such as the identification of cultural isolates and the results of antimicrobial susceptibility tests (AST).

At 208, the message exchange 110 may send, to the analysis engine 120, the clinically significant data to enable one or more downstream tasks. In some example embodiments, clinically significant data extracted from one or more actionable messages may enable one or more downstream tasks and/or decisions. Examples of downstream tasks and/or decisions, which may be performed at the analysis engine 120, may include the collection of workflow statistics, resource allocation, and/or the like.

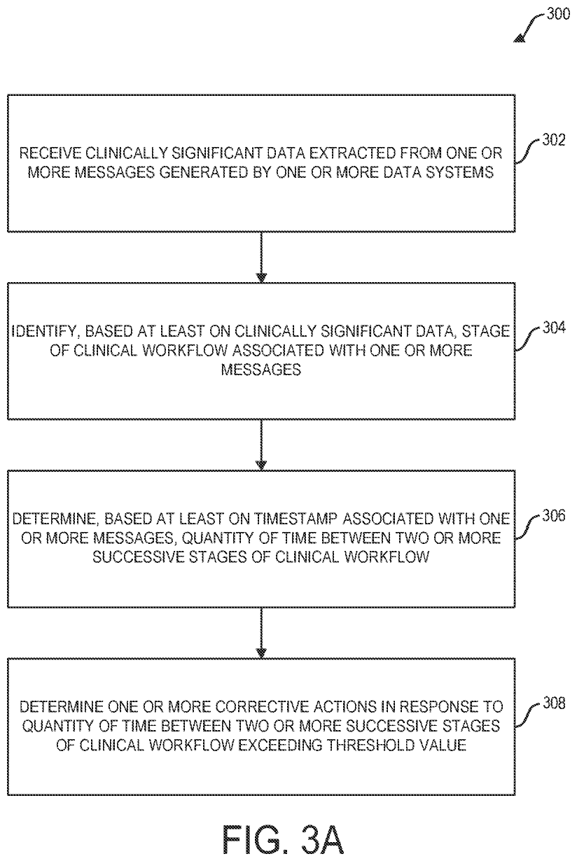

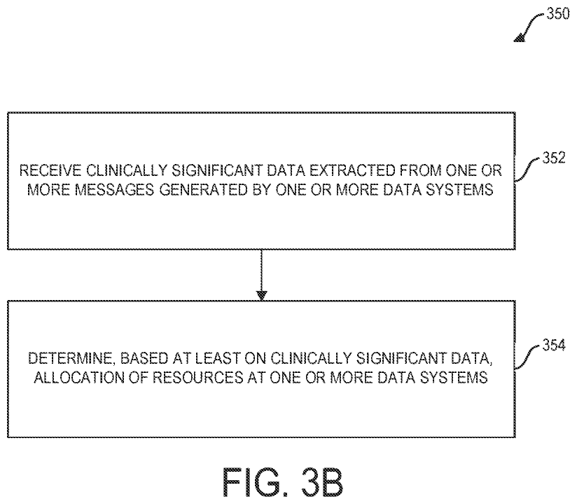

FIG. 3A depicts a flowchart illustrating an example of a process 300 for machine learning based clinical workflow analysis, in accordance with some example embodiments. Referring to FIGS. 1-2 and 3A, the process 300 may be performed by the analysis engine 120.

At 302, the analysis engine 120 may receive, from the message exchange 110, clinically significant data extracted from one or more messages generated by one or more data systems. For example, for the microbial testing workflow associated with the laboratory information system 145a, actionable messages conveying the clinically significant data may indicate a start of a culturing process for a microbe, a gram positive or gram negative identification for the microbe, a species and/or organism identification for the microbe, an antimicrobial susceptibility of the microbe, and/or the like.

At 304, the analysis engine 120 may identify, based on the clinically significant data, a stage of a clinical workflow associated with each of the one or more messages. For example, for the microbial testing workflow associated with the laboratory information system 145a, stages of the workflow may include inoculation, incubation, plate imaging, culture reading, result reporting, and/or the like.

At 306, the analysis engine 120 may determine, based at least on a timestamp associated with the one or more messages, a quantity of time between two or more successive stages of the clinical workflow. In some example embodiments, the analysis engine 120 may determine various workflow statistics. For the microbial testing workflow, for example, the workflow statistics may be determined based at least on a volume and/or a timing of the messages from various stages of the microbial testing workflow. Examples of clinical workflow statistics may include various metrics such as a turnaround time (TAT) indicating a distribution of time elapsed between successive stages of a workflow such as the microbial testing workflow associated with the laboratory information system 145a. The analysis engine 120 may also identify trends and/or establish benchmarks to enable a comparison between different medical devices, clinical facilities, and automation platforms. These metrics, trends, and/or benchmarks may enable a detection of systematic inefficiencies and bottlenecks across devices, facilities, and automation platforms.

At 308, the analysis engine 120 may determine one or more corrective actions in response to the quantity of time between the two or more successive stages of the clinical workflow exceeding a threshold value. For example, the analysis engine 120 may identify, based at least on a timestamp associated with the actionable messages, bottlenecks and/or systematic inefficiencies present in the microbial testing workflow in which one or more laboratory activities and/or stages of the workflow are associated with an above threshold delay. In response to detecting a bottleneck and/or a systematic inefficiency present in the microbial testing workflow, the analysis engine 120 may determine one or more corrective actions including, for example, modifying the scheduling of the laboratories activities, adjusting the allocation of resources associated with the laboratories activities, and/or the like.

FIG. 3B depicts a flowchart illustrating another example of a process 350 for machine learning based resource allocation, in accordance with some example embodiments. Referring to FIGS. 1-2 and 3B, the process 350 may be performed by the analysis engine 120.

At 352, the analysis engine 120 may receive from the message exchange 110, clinically significant data extracted from one or more messages generated by one or more data systems. As noted, actionable messages output by the one or more data systems 140 may convey clinically significant data, which are associated with additional tasks. For example, for the microbial testing workflow associated with the laboratory information system 145a, actionable messages conveying the clinically significant data may indicate a start of a culturing process for a microbe, a gram positive or gram negative identification for the microbe, a species and/or organism identification for the microbe, an antimicrobial susceptibility of the microbe, and/or the like.

At 354, the analysis engine 120 may determine, based at least on the clinically significant data, an allocation of resources at the one or more data systems. For example, in response to the clinically significant data indicating the presence of a microbe susceptible to an antimicrobial, for example, in more than a threshold quantity of specimen encountered at the laboratory information system 145a, the resource controller 125 at the analysis engine 120 may determine to allocate a corresponding quantity of the antimicrobial (and/or other resources). Resource allocation may also be performed based on the predicted sequence and timing of the messages originating from the laboratory information system 145a. For instance, the clinically significant data extracted from the actionable messages may also include an expected sequence and timing of the messages originating from the laboratory information system 145a. The resource controller 125 may schedule, based at least on a timestamp of a message indicating the start of a culturing process for a microbe, a suitable quantity of laboratory resources at the appropriate times for the subsequent stages of the microbial testing workflow (e.g., the gram positive or gram negative identification for the microbe, the species and/or organism identification for the microbe, the antimicrobial susceptibility of the microbe, and/or the like).

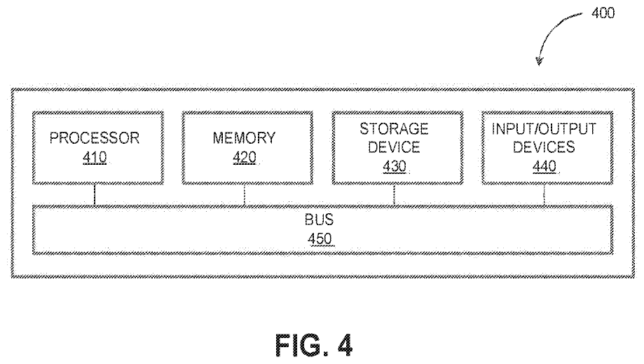

FIG. 4 depicts a block diagram illustrating a computing system 400 consistent with implementations of the current subject matter. Referring to FIGS. 1-2, 3A-B and 4, the computing system 400 can be used to implement the message exchange 110, the analysis engine 120, and/or any components therein.

As shown in FIG. 4, the computing system 400 can include a processor 410, a memory 420, a storage device 430, and input/output device 440. The processor 410, the memory 420, the storage device 430, and the input/output device 440 can be interconnected via a system bus 450. The processor 410 is capable of processing instructions for execution within the computing system 400. Such executed instructions can implement one or more components of, for example, the message exchange 110 and/or the analysis engine 120. In some example embodiments, the processor 410 can be a single-threaded processor. Alternatively, the processor 410 can be a multi-threaded processor. The processor 410 is capable of processing instructions stored in the memory 420 and/or on the storage device 430 to display graphical information for a user interface provided via the input/output device 440.

The memory 420 is a computer readable medium such as volatile or non-volatile that stores information within the computing system 400. The memory 420 can store data structures representing configuration object databases, for example. The storage device 430 is capable of providing persistent storage for the computing system 400. The storage device 430 can be a floppy disk device, a hard disk device, an optical disk device, a tape device, a solid-state device, and/or any other suitable persistent storage means. The input/output device 440 provides input/output operations for the computing system 400. In some example embodiments, the input/output device 440 includes a keyboard and/or pointing device. In various implementations, the input/output device 440 includes a display unit for displaying graphical user interfaces.

According to some example embodiments, the input/output device 440 can provide input/output operations for a network device. For example, the input/output device 440 can include Ethernet ports or other networking ports to communicate with one or more wired and/or wireless networks (e.g., a local area network (LAN), a wide area network (WAN), the Internet).

In some example embodiments, the computing system 400 can be used to execute various interactive computer software applications that can be used for organization, analysis and/or storage of data in various formats. Alternatively, the computing system 400 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 440. The user interface can be generated and presented to a user by the computing system 400 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data can be forwarded to a “remote” device or location,” where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

One or more aspects of the artificial intelligence described may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a desired device state. In such instance, the training may include receiving the three characteristics as inputs to the model and adjusting the characteristics of the model such that for each set of three characteristics, the output device state matches the desired device state associated with the historical data.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions which, when executed by the at least one data processor, cause the system to perform operations comprising:
receiving, at a machine learning model, from one or more laboratory information systems, one or more messages for a patient as at least one input value to the machine learning model, wherein the one or more messages provide information about a clinical workflow;
based on the machine learning model receiving the one or more messages as the at least one input value to the machine learning model, outputting, by the machine learning model, at least one output value indicating that the one or more messages are actionable;
extracting, from the one or more messages, clinically significant data; and
controlling, based at least on the clinically significant data, at least one medical device associated with the patient to perform one or more tasks.

2. The system of claim 1, wherein the one or more tasks include:
identifying, based at least on the clinically significant data, a stage of the clinical workflow associated with the one or more messages;
determining, based at least on a timestamp associated with the one or more messages, a quantity of time between two or more successive stages of the clinical workflow; and
in response to the quantity of time between the two or more successive stages of the clinical workflow exceeding a threshold value, determining one or more corrective actions.

3. The system of claim 2, wherein the one or more corrective actions include one or more of: 1) modifying a scheduling of one or more activities associated with the clinical workflow; or 2) adjusting an allocation of resources associated with the one or more activities.

4. The system of claim 2, wherein the stage of the clinical workflow includes a start of a culturing process for a microbe, a gram positive or gram negative identification for the microbe, a species or organism identification for the microbe, or an antimicrobial susceptibility of the microbe.

5. The system of claim 1, wherein the clinical workflow comprises a microbial testing workflow or a virology assay.

6. The system of claim 1, wherein the one or more tasks include:
determining, based at least on the clinically significant data, to allocate a resource to the one or more laboratory information systems.

7. The system of claim 6, wherein the resource includes an antimicrobial based at least on the clinically significant data indicating a presence of a microbe susceptible to the antimicrobial.

8. The system of claim 6, wherein to allocate the resource comprises:
determining, based at least on the clinically significant data, a subsequent stage of the clinical workflow and a time for the subsequent stage of the clinical workflow; and
scheduling, in accordance with the time of the subsequent stage of the clinical workflow, a quantity of resources required for the subsequent stage of the clinical workflow.

9. The system of claim 1, wherein extracting the clinically significant data comprises inputting the one or more messages into one or more of the machine learning model or a different machine learning model, the one or more of the machine learning model or the different machine learning model outputting the clinically significant data.

10. The system of claim 1, wherein the one or more messages are determined to be actionable in response to more than a threshold quantity of data in the one or more messages being tagged as clinically significant by the machine learning model.

11. The system claim 1, wherein receiving the one or more messages comprises receiving a sequence of messages, and wherein the at least one output value indicates whether the sequence of messages are actionable.

12. The system of claim 11, wherein the at least one output value indicates that a message of the sequence of messages is associated with a first actionable event, and wherein the at least one output value further indicates that the sequence of messages are associated with a second actionable event.

13. The system of claim 12, wherein the machine learning model determines the message to be actionable as part of the sequence of messages.

14. The system of claim 1, wherein the at least one medical device comprises one or more of a diagnostic device, an infusion pump, a dispensing cabinet, or a wasting station.

15. The system of claim 1, wherein controlling the at least one medical device comprises transmitting, to the at least one medical device, at least one message to adjust one or more of an operational state or a functional element of the at least one medical device.

16. The system of claim 1, wherein the machine learning model comprises one or more of: a regression model, an instance-based model, a regularization model, a decision tree, a Bayesian model, a clustering model, an associative model, a neural network, a deep learning model, a dimensionality reduction model an ensemble model, a recurrent neural network (RNN), a hidden Markov model, a conditional random field (CRF) model, or a gated recurrent unit (GRU).

17. A computer-implemented method, comprising:
receiving, at a machine learning mode, from one or more laboratory information systems, one or more messages for a patient as at least one input value to the machine learning model, wherein the one or more messages provide information about a clinical workflow;
based on the machine learning model receiving the one or more messages as the at least one input value to the machine learning model, outputting, by the machine learning model, at least one output value indicating that the one or more messages are actionable;
extracting, from the one or more messages, clinically significant data; and
controlling, based at least on the clinically significant data, at least one medical device associated with the patient to perform one or more tasks.

18. The method of claim 17, wherein the clinical workflow comprises a microbial testing workflow or a virology assay.

19. A non-transitory computer readable medium storing instructions, which when executed by at least one data processor, cause the at least one data processor to perform operations comprising:
receiving, at a machine learning model, from one or more laboratory information systems, one or more messages for a patient as at least one input value to the machine learning model, wherein the one or more messages provide information about a clinical workflow;
based on the machine learning model receiving the one or more messages as the at least one input value to the machine learning model, outputting, by the machine learning mode, at least one output value indicating that the one or more messages are actionable;
extracting, from the one or more messages, clinically significant data; and
controlling, based at least on the clinically significant data, at least one medical device associated with the patient to perform one or more tasks.

20. The non-transitory computer readable medium of claim 19, wherein the clinical workflow comprises a microbial testing workflow or a virology assay.
